(19) United States
(12) Patent Application Publication
(10) Pub. No.: US 2025/0161350 A1
(43) Pub. Date: May 22, 2025

(54) METHODS FOR USING CERIUM OXIDE NANOPARTICLES FOR MACROPHAGE-MEDIATED EFFICACY IN RESPIRATORY SYNCYTIAL VIRAL INFECTION

(71) Applicant: UNIVERSITY OF PITTSBURGH- OF THE COMMONWEALTH SYSTEM OF HIGHER EDUCATION, Pittsburgh, PA (US)

(72) Inventors: Shilpa Sant, Oak Park, IL (US); Akhil Pravinkumar Patel, Cambridge, MA (US); Vinayak Sant, Oak Park, IL (US); Kerry McGarr Empey, Mars, PA (US); Jessica L. Kosanovich, Cranberry Township, PA (US)

(21) Appl. No.: 18/842,461

(22) PCT Filed: Mar. 3, 2023
(86) PCT No.: PCT/US2023/014471

§ 371 Date: Aug. 29, 2024

Related U.S. Application Data
(60) Provisional application No. 63/316,576, filed on Mar. 4, 2022.

(51) Int. Cl. A61K 33/24; A61K 9/00; A61K 9/14; A61P 31/14

(52) U.S. Cl. CPC A61K 33/24; A61K 9/0043; A61K 9/14; A61P 31/14

(57) ABSTRACT

The invention relates to shape-specific cerium oxide nanoparticles (CNPs). methods of preparing CNPs. and methods of using CNPs to treat and/or inhibit and/or reduce and/or reverse the complications of respiratory syncytial virus (RSV) disease and other infections that cause pathologic immune responses. through the balance of favorable immunomodulation and reduced lung immunopathology. CNPs have the capability to switch between Ce³⁺ and Ce⁴⁺ oxidation states (e.g., to scavenge reactive oxygen species). The shape-specific CNPs are synthesized into various shapes and sizes. These properties offer an opportunity to utilize CNPs to modulate macrophage phenotypes along the spectrum of M1 and M2 phenotypes to combat RSV infection and other infections that cause pathologic immune responses.

### CROSS-REFERENCE TO RELATED APPLICATION

This application claims priority under 35 U.S.C. § 119(e) to U.S. Provisional Patent Application Ser. Nos. 63/316,576, filed Mar. 4, 2022, entitled “METHODS FOR USING CERIUM OXIDE NANOPARTICLES FOR MACROPHAGE-MEDIATED EFFICACY IN RESPIRATORY SYNCYTIAL VIRAL INFECTION”, which is herein incorporated by reference.

### FIELD OF THE INVENTION

The invention relates to cerium oxide nanoparticles, and related systems and methods, to prevent and/or treat the complications of respiratory viruses and/or diseases, such as respiratory syncytial virus (RSV) disease, and/or other infections that cause pathologic immune responses, through the balance of favorable immunomodulation and reduced lung immunopathology.

### BACKGROUND

Respiratory viruses are a major cause of mortality worldwide, with an estimated 2.7 million deaths reported in a single year. As of Sep. 13, 2021, the World Health Organization reported over 4.6 million deaths due to SARS-CoV-2 (coronavirus-2) alone, a number which is continuing to rise. This emerging pathogen joins the ranks of other deadly viruses, such as influenza, which claims the lives of up to 200,000 people annually, and respiratory syncytial virus (RSV), which kills approximately 18,000 people each year. In the United States, annual RSV infection leads to ˜1.5 million outpatient visits among children less than five years of age with up to 125,000 estimated RSV-related hospitalizations in children less than one year of age. Respiratory syncytial virus (RSV) is the single most common cause of viral bronchiolitis among children worldwide. Despite the global burden of the disease, there is no effective treatment or vaccine for RSV disease.

Severe RSV infection is associated with bronchiolar obstruction, air trapping, and emphysema due to excess mucus secretion, apoptotic cellular debris, and inflammatory cell recruitment. Furthermore, RSV infection upregulates growth arrest specific-6 (Gas6), which induces conversion of macrophages to an M2-like phenotype, thereby increasing the susceptibility of patients to secondary bacterial infections such as pneumococcal infection. While each pulmonary virus elicits a unique immune response to contain viral spread, a common factor among most pulmonary viruses is the inherent cost to mounting a robust immune response—tissue damage and immunopathology. Thus, a therapeutic strategy that mitigates damaging immune responses to viruses without sacrificing antiviral activity is desperately needed.

Mounting evidence suggests that optimum levels of reactive species (oxygen and nitrogen) are important for viral clearance. Notably, a low level of reactive oxygen species (ROS) triggers a signaling cascade that modulates redox-sensitive genes regulating immune and proinflammatory response, whereas a high level of ROS induces inflammation and tissue damage. ROS is generated by cellular organelles during metabolism of O2 or by membrane bound enzymes such as the nicotinamide adenine dinucleotide phosphate (NADPH) oxidase (Nox) family. In the lung, Nox2 functions by producing ROS in alveolar macrophages during infection or inflammation. During RSV infection, the virus induces oxidative stress, which is further enhanced by RSV-induced transcriptional inhibition of antioxidant enzymes. A hallmark of severe pulmonary viral infections is an increase in ROS. As such, reactive oxygen species are a potential therapeutic target. Several studies have demonstrated that antioxidant treatment with butylated hydroxyanisole (BHA) or resveratrol and the mitochondrial ROS scavenger mitoquinone mesylate, reduced levels of peroxidation products and ameliorated clinical illness.

Several research approaches utilizing soluble proteins/cytokines have focused on macrophage modulation to mitigate the underlying condition caused by RSV infection as well as viral clearance. However, most of these approaches face risk of inducing excessive inflammation in the infant airways. Therefore, less potent candidates are sought, which can be smart to stop or reduce their macrophage modulation potency.

Thus, a therapeutic strategy that mitigates damaging immune responses to viruses without sacrificing antiviral activity is needed.

Cerium oxide nanoparticles (CNPs) are known to scavenge oxygen species through their superoxide dismutase (SOD)-mimetic or antioxidant catalase-mimetic ability, through trivalent cerium (Ce3+). The ROS-scavenging activity of CNPs is directly proportional to oxygen vacancies on their surface (Ce3+/Ce4+ state). Moreover, these crystalline nanoparticles are proven safe in preclinical studies, and shown to be tolerated up to 100 mg/kg for ten days in male rats. CNPs have demonstrated about nine-fold greater antioxidant activity compared to commercial antioxidant Trolox, garnering interest in the therapeutic potential of CNPs to modulate oxidative stress in various diseases.

In RSV disease, it is important to balance levels of reactive species (oxygen and nitrogen) for mounting a robust immune response necessary to contain the viral spread without eliciting tissue damage and immunopathology. To this end, the present invention describes the use of shape-specific CNPs in modulating macrophage phenotype and innate cellular responses in respiratory viruses and/or diseases, such as RSV disease, and/or other infections that cause pathologic immune responses. Additionally, the shape-specific CNPs can be combined with other drugs or vaccines as adjuvants to achieve better therapeutic outcomes in RSV disease or other infections with similar pathologies.

### SUMMARY OF THE INVENTION

In one aspect, the invention provides a pharmaceutical composition that includes shape-specific cerium oxide nanoparticles, wherein the cerium oxide nanoparticles switch between the Ce3+ and Ce4+ oxidation states to scavenge reactive oxygen species: and a pharmaceutical carrier.

In certain embodiments, the shape-specific cerium oxide nanoparticles have a shape selected from the group consisting of sphere, cube, rod or a combination thereof.

The pharmaceutical composition may be in a dosage form selected from the group consisting of a parenteral dosage form, an oral dosage form, and combination thereof. In certain embodiments, the dosage form is selected from the group consisting of a tablet, capsule, dry powder, gel, film, suspension, solution, or combination thereof.

In certain embodiments, the pharmaceutical carrier is selected from the group consisting of a solvent, a polymer, a nanoparticle, a liposome, a lipoprotein, a gel, a sugar or sugars, protein or other matrix, carriage device, and combinations thereof.

The shape-specific cerium oxide nanoparticles may be effective to modulate macrophage phenotype and innate cellular responses in respiratory syncytial virus disease.

In certain embodiments, the shape-specific cerium oxide nanoparticles in the shape of a sphere have an average diameter from about 8 to about 12 micrometer in dry form and 365 to about 557 micrometer in hydrodynamic form and, in certain embodiments, the average diameter is about 11.3 nm.

In certain embodiments, the shape-specific cerium oxide nanoparticles in the shape of a cube have an average dimension from about 24 to about 40 micrometer in dry form and 196 to 200 micrometer in hydrodynamic form and, in certain embodiments, the average dimension is about 19.3 nm.

In another aspect, the invention provides a method of treating respiratory syncytial virus disease or other infection that causes a pathologic immune response. The method includes administering shape-specific cerium oxide nanoparticles to a patient, wherein the cerium oxide nanoparticles switch between the Ce3+ and Ce4+ oxidation states.

In certain embodiments the shape-specific cerium nanoparticles are selected from the group consisting of sphere cerium oxide nanoparticles, cube cerium nanoparticles, and combinations thereof.

The administering step may include administering a pharmaceutical composition including the shape-specific cerium oxide nanoparticles, and a pharmaceutical carrier.

The administering step may include applying ex-vivo the shape-specific cerium oxide nanoparticles to a patient, and the applying step may include cell priming for cell therapy.

The route of delivery of the pharmaceutical composition can be by injection, oral, sublingual, buccal, transdermal, or nasal.

In another aspect, the invention provides a method of preparing shape-specific cerium oxide nanoparticles. The method includes (i) preparing sphere-shaped cerium oxide nanoparticles including synthesizing cerium oxide by an ultrasonication reaction between cerium nitrate and sodium hydroxide, including forming colloidal nanocrystals in a first step of the reaction, and adsorbing hydroxyl ions to form spherical nanocrystals, or (ii) preparing cube-shaped cerium oxide nanoparticles including synthesizing cerium oxide by a hydrothermal reaction between cerium nitrate and sodium hydroxide, in a hydrothermal reactor, including oxidizing intermediate nanocrystals selected from the group consisting of nanorods and nanotubes; and coarsening the nanocrystals to reach a stable crystal state forming cube morphology. Additional steps may include adding and surface adsorbing methoxy polyethylene glycol during the synthesizing step to provide steric hindrance to prevent agglomerate formation.

### BRIEF DESCRIPTION OF THE DRAWINGS

FIGS. 1(a1), 1(b1), 1(a2), 1(b2), 1(a3), 1b3), 1(a4), 1(b5), 1(a5), 1(b5), 1(c) and 1(d) show the synthesis and physicochemical characterization of cerium oxide nanoparticles (CNPs), according to certain embodiments of the invention; the figures include a schematic representation of synthesis of sphere CNPs using an ultrasonication method (FIG. 1(a1)) and synthesis of cube CNPs using a hydrothermal method (FIG. 1(b1)); transmission electron microscopy images of sphere-shaped CNPs (FIG. 1(a2)) and cube-shaped CNPs (FIG. 1(b2)) with scale bars representing 100 nm; effect of buffer (water, PBS, or HEPES) on hydrodynamic size and zeta potential of sphere CNPs (FIG. 1(a3)) and cube CNPs (FIG. 1(b3)); effect of pH on hydrodynamic size and zeta potential of sphere CNPs (FIG. 1(a4)) and cube CNPs (FIG. 1(b4)); effect of serum on hydrodynamic size and zeta potential of sphere CNPs (FIG. 1(a5)) and cube CNPs (FIG. 1(b5)); SDS-PAGE of separated proteins from sphere CNPs and cube CNPs (FIG. 1(c)); and x-ray diffraction spectra of sphere CNPs and cube CNPs with crystallite quantified using Scherrer equation (FIG. 1(d)). ;p FIGS. 2(a), 2(b1), 2(c1), 2 b2), 2(c2), 1(b3), 2b4), 2(c3), 2(c4), 2(b5), 2(c5), and 2(d) show that shape-specific CNPs are cytocompatible and are readily taken up by J774A.1 macrophages in vitro, according to certain embodiments of the invention; the figures include percentage cell viability of J774A.1 macrophages after 24 hours incubation with sphere CNPs and cube CNPs at various concentrations (FIG. 2(a)); schematic representation of in vitro uptake of sphere CNPs (FIG. 2(b1)) and cube CNPs (FIG. 2(c1) by J774A.1 macrophages: DIC images after 6 hours of incubation with sphere CNPs (FIG. 2(b2)) and cube CNPs (FIG. 2(c2)) with scale bars representing 100 μm: confocal images with multiple z-stacks showing uptake of FITC-sphere (FIGS. 2(b3), (b4)) and FITC-cube CNPs (FIGS. 2(c3), (c4)) at different magnifications, wherein green channel indicates FITC-tagged CNPs and blue channel indicates HOECHST-stained nuclei of macrophages: and confocal images with DIC channel showing macrophage boundaries and sphere CNPs (FIG. 2(b5)) and cube CNPs (FIG. 2(c5)); confocal image analysis further showed significantly higher mean fluorescence intensity (MFI) for cube CNPs compared to sphere CNPs confirming qualitative observations and indicating higher macrophage uptake of cube CNPs (FIG. 2(d)).

FIGS. 3, 3(a), 3(b), 3(c) and 3(d) show CNP shape triggers differential ROS and RNS levels in RSV-infected macrophages, according to certain embodiments of the invention; including measurement of reactive oxygen species (ROS) using CellROX assay following RSV infection with or without CNPs for 6 or 12 hours (FIGS. 3, 3(a) and 3(b)); measurement of reactive nitrogen species (RNS) using Griess reagent assay following respiratory syncytial virus (RSV) infection with or without CNPs for 6 or 12 hours (FIGS. 3(c) and 3(d)); a one-way ANOVA with Tukey's multiple comparison post-hoc test was used to compare groups, * p<0.05; **p<0.01; ***p<0.001; p<0.0001 when compared to RSV-infected macrophages.

FIGS. 4(a), 4(b), 4(c), 4(d), 4(c), 4(f), 4(g), 4(h), 4(i), 4(j) and 4(k) show that the shape of CNPs alters macrophage phenotypes in presence of RSV infection, according to certain embodiments of the invention: the figures include J774A. 1 macrophages infected with RSV Line 19 at a multiplicity of infection (MOI) of 3 for 3 hours prior to treatment with sphere or cube CNPs, at 6 hours post-nanoparticle treatment, percent of macrophages with the phenotypes CD86+CD206-(FIG. 4(a)), CD86-CD206+ (FIG. 4(b)), CD80+CD206− (FIG. 4(d)) and CD80-CD206+ (FIG. 4(e)) were quantified via flow cytometry; ratio of CD86+:CD206+ (FIG. 4(c)) and CD80+:CD206+ (FIG. 4(f)) macrophages were calculated for each group; at 24 hours post-nanoparticle treatment, TNFα (FIG. 4(g)), IL-10 (FIG. 4(h)), and IL-12p70 (FIG. 4(j)) cytokine levels were measured from cell supernatants via Luminex; ratios of TNFα:IL-10 (i) and IL-12p70:IL-10 (FIG. 4(k)) were calculated for each group: data is represented as mean +/−SEM; a one-way ANOVA with Dunnett's multiple comparison post-test was used to compare groups with RSV infection and each treatment condition, *p<0.05, **p<0.01, ***p<0.001, and ****p<0.0001.

FIGS. 5(a), 5(b) and 5(c) show that intranasal administration of shape-specific CNPs is safe; 8-week-old Balb/c mice were intranasally infected with 5×10⁵ PFU/gram of RSV Line 19, according to certain embodiments of the invention: at 1- and 3-days post-infection, mice received 55 μg/100 μl dose of sphere or cube CNPs or were administered 10 mM HEPES as a vehicle control (FIG. 5 a)); weight was measured daily before and throughout infection and is represented as % of original weight (FIG. 5(b)); at 4 days post-infection, bronchoalveolar lavage cells (BAL) were assessed for viability (FIG. 5(c)); data is represented as mean +/−SEM; a two-way ANOVA or a one-way ANOVA with Tukey's multiple comparison post-test was used to compare groups.

FIGS. 6(a), 6(b), 6(c), 6(d), 6(c), 6(f), 6(g), 6(h), 6(i), and 6(k) show that the shape of CNPs leads to differential uptake by immune cells in murine lungs, according to certain embodiments of the invention: the figures show mice were infected and treated as previously described (FIG. 6(a)); at 4 dpi, total alveolar macrophages (FIG. 6(b)), monocytes (FIG. 6(c)), dendritic cells (FIG. 6d)), and neutrophils (FIG. 6(e)) were quantified in the BAL of infected animals via flow cytometry; percent of nanoparticle uptake by alveolar macrophages (FIG. 6(f)), monocytes (FIG. 6(g)), dendritic cells (FIG. 6(h)), and neutrophils (FIG. 6(i)) was measured via flow cytometry; alveolar macrophages were imaged to assess uptake of sphere or cube CNPs (FIG. 6 (k)) data is represented as mean +/−SEM; a one-way ANOVA with Tukey's multiple comparison post-test was used to compare groups. *p<0.05, ***p<0.001, and ****p<0.0001.

FIGS. 7(a), 7(b), 7(c) and 7(d) show that CNP treatment activates aleveolar macrophages, according to certain embodiments of the invention: mice were infected and treated as previously described: the figures show total CD86+ (FIG. 7(a)) and MHCII+ (FIG. 7(c)) wherein alveolar macrophages were quantified via flow cytometry in the BAL of infected animals: mean fluorescence intensity (MFI) of CD86 (FIG. 7(b)) and MHCII (FIG. 7(d)) on alveolar macrophages was determined via flow cytometry: statistical significance was calculated using an unpaired t-test between groups, **p<0.01.

### DETAILED DESCRIPTION OF PREFERRED EMBODIMENTS

While the general inventive concepts are susceptible of embodiment in many forms, there are shown in the drawings, and will be described herein in detail, specific embodiments thereof with the understanding that the present disclosure is to be considered an exemplification of the principles of the general inventive concepts. Accordingly, the general inventive concepts are not intended to be limited to the specific embodiments illustrated herein.

It is also to be understood that the terminology used herein is for the purpose of describing particular embodiments only, and is not intended to be limiting.

The articles “a” and “an” are used herein to refer to one or more than one (i.e., to at least one) of the grammatical object of the article. By way of example, “a nanoparticle” means one nanoparticle or more than one nanoparticle.

“About” as used herein when referring to a measurable value such as an amount, a temporal duration, and the like, is meant to encompass variations of ±5%, preferably ±1%, and still more preferably ±0.1% from the specified value, as such variations are appropriate to perform the disclosed methods.

As used herein the term “patient” or “user” means a member of the animal kingdom, including, but not limited to, a human.

As used herein, the term “therapeutically effective amount” refers to that amount of any of the present compounds, e.g., cerium oxide nanoparticles, or compositions required to bring about a desired effect in a patient. The desired effect will vary dependent on the desired therapeutic response. As will be understood by one skilled in the art, a therapeutically effective amount of said compounds or compositions is administered by any means known in the art, including but not limited to, injection, intravenously, parenterally, orally, bucally, transdermally, nasally, or where appropriate, topically.

In addition, for ease of description, there is disclosed herein information relating to respiratory syncytial virus (RSV) disease, however, it is contemplated and intended by the inventors that the invention and application or administration of the invention are not limited to RSV disease, and are equally applicable to other respiratory viruses and diseases, and/or other infections that cause one or more pathologic immune response, known in the art. Thus, RSV is merely exemplary of the viruses and diseases encompassed by the treatment systems and methods of the invention.

Additionally, for ease of description, there is disclosed herein information relating to sphere and cube cerium oxide nanoparticles, however, it is contemplated and intended by the inventors that the invention and application or administration of the invention are not limited to only sphere-shaped and cube-shaped cerium oxide nanoparticles, and are equally applicable to other shaped cerium oxide nanoparticles, such as, but not limited to, rod shaped cerium oxide nanoparticles. Thus, the sphere and cube shapes are merely exemplary of the various shapes encompassed by the treatment systems and methods of the invention.

This invention describes shape-specific cerium oxide nanoparticles (CNPs), methods of preparing CNPs, and methods of using CNPs to treat and/or inhibit and/or reduce and/or reverse the complications of RSV disease, and other respiratory viruses and/or diseases, and/or other infections that cause pathologic immune responses, through the balance of favorable immunomodulation and reduced lung immunopathology. CNPs have the capability to switch between Ce3+ and Ce4+ oxidation states (e.g., to scavenge reactive oxygen species) and are known for their self-regenerative, and reactive species scavenging properties, as well as immune-modulating effects. Moreover, CNPs are synthesized into various shapes and sizes, e.g., spheres, cubes, and rods. These properties offer an opportunity to utilize CNPs to modulate macrophage phenotypes along the spectrum of M1 and M2 phenotypes to combat RSV infection, and other respiratory viruses and/or diseases, and/or other infections that cause pathologic immune responses.

The inventors have surprisingly demonstrated that the reactive oxygen species (ROS)-scavenging activity of CNPs mitigates oxidative stress-induced calcification in patient-derived primary valve interstitial cells. Additionally, the inventors have surprisingly shown that such ROS-scavenging activity of CNPs is dependent on their shape. Nanoparticle shape influences cellular uptake, as demonstrated by the enhanced specificity of cancer cell lines for rod-shaped ligand-coated nanoparticles compared to their spherical counterpart. The invention describes the synthesis of shape-specific CNPs, and the use of shape-specific CNPs in modulating macrophage phenotype and innate cellular responses in RSV disease.

In certain embodiments, cerium oxide is synthesized into CNPs having one or more shapes. The shapes of the CNPs vary and include, but are not limited to, cubes and/or spheres. Specifically, the inventors have found that CNPs in the shape of a cube significantly reduce RSV-induced ROS levels without affecting reactive nitrogen species (RNS) levels, while CNPs in the shape of a sphere increase RSV-induced RNS levels with minimal effect on ROS levels. In addition, cube CNPs drive an M1 phenotype in RSV-infected macrophages in vitro by increasing macrophage surface expression of CD80 and CD86 with a concomitant increase in TNFα and IL-12p70, while simultaneously decreasing M2 CD206 expression. Furthermore, cube CNPs preferentially accumulate in murine alveolar macrophages and induce activation, avoiding enhanced uptake and activation of inflammatory cells such as neutrophils, which are associated with RSV-mediated inflammation.

Suitable methods for synthesizing the shape-specific CNPs include conventional nanoparticle synthesis methods known in the art. For example, sphere-shaped CNPs are synthesized by an ultrasonication method including the reaction of cerium nitrate and sodium hydroxide, whereby colloidal nanocrystals are formed in a first step of the reaction, and then adsorb hydroxyl ions to form spherical nanocrystals. In certain embodiments, this synthesis method is facilitated by weak surface adsorption of methoxy polyethylene glycol (mPEG5000) that is added during the synthesis, which provides steric hindrance to prevent agglomerate formation. The sphere CNPs have an average diameter from about 2 nm to about 100 nm. In certain embodiments, the sphere CNPs have an average diameter from about 8 to about 12 micrometers in dry form, and from about 365 to about 557 micrometers in hydrodynamic form. In certain other embodiments, the average diameter of sphere-shaped CNPs is about 11.3 nm.

Suitable methods for synthesizing the cube-shaped CNPs include a hydrothermal method including the reaction of cerium nitrate and sodium hydroxide. For example, nanocube-shaped CNPs are synthesized by reacting cerium nitrate and sodium hydroxide in a hydrothermal reactor at a high temperature achieved under high pressure (e.g., for about 24 hours). Various high temperature and high pressure values are suitable for use. This method allows oxidation of intermediate nanocrystals such as nanorods and nanotubes, which further coarsen to reach a stable crystal state forming cube morphology. The cube CNPs have an average dimension from about 2 nm to about 100 nm. In certain embodiments, the cube CNPs have an average dimension from about 24 to about 40 micrometers in dry form, and from about 196 to about 200 micrometers in hydrodynamic form. In certain other embodiments, the average dimension is about 19.3 nm.

Increased ROS level is one of the hallmarks of respiratory viruses and/or diseases, such RSV disease, and/or other infections that cause pathologic immune responses, which leads to tissue damage and airway inflammation. It has been found that shape-specific CNPs are effective to alter redox microenvironment. Both of the sphere-and cube-shaped CNPs modulate reactive oxygen and nitrogen species in J774A1 macrophages in the absence or presence of RSV infection. The inventors have found that the administration of cube CNPs in a 12-hour treatment of RSV-infected macrophages is effective to significantly reduce a RSV-induced increase in ROS levels. As a result, cube CNPs have the capability to effectively reduce tissue damage through reduction of ROS levels in RSV-infected macrophages. Similar treatment results were found for sphere CNP treatment in RSV-infected macrophages, e.g., both sphere and cube CNPs have the capability to decrease RSV-induced ROS production.

RNS are known to be the fulcrum that shifts the macrophage polarity from pro-inflammatory (M1) to regenerative (M2) states. Optimal extracellular RNS level is important to enable cytotoxic activity of macrophages. Unlike ROS generated within the cytoplasm of the macrophages, RNS is generated extracellularly from rapid diffusion of nitric oxide (NO) out of cell membrane. Nitric oxide reacts with other oxygen species in the extracellular medium producing RNS, which are relatively more stable compared to short-lived nitric oxide. Such extracellular RNS, together with induced nitrous oxide synthase (iNOS) enzyme, provide protection and eradicate pathogens such as mycobacterium tuberculosis. RSV infection is known to alter homeostatic levels of NO and RNS primarily through iNOS induction. The inventors have found that sphere CNP treatment of RSV-infected macrophages effectively. significantly increases RSV-induced RNS levels. Cube CNP treatment, however, was found not to alter RSV-induced RNS levels, further highlighting shape-driven differential responses in modulating extracellular RNS levels in RSV-infected macrophages. Such shape-driven differential response in RNS modulation is also evident in virus-free macrophages treated with CNPs; specifically, sphere CNPs increase and cube CNPs reduce RNS levels significantly in virus-free macrophages compared to untreated RSV-infected macrophages.

The reduction of ROS levels and optimal RNS levels are beneficial to prevent tissue damage in lungs. It has been found that RSV infection alone fails to increase the frequency of M1 macrophages, however, treatment of uninfected and RSV-infected macrophages with sphere and cube CNPs increases the frequency of M1 macrophages (e.g., CD86+ CD206-J774A.1 macrophages). Although, the inventors found that sphere- and cube-shaped CNPs reduce the frequency of M2 macrophages (e.g., CD86− CD206+ macrophages) in both uninfected and RSV-infected macrophages relative to RSV infection alone. CNPs have the capability to alter the surface expression of M1 and M2 macrophage markers. Thus, CNP shape influences RSV-associated macrophage activation with cube CNPs favoring a more anti-viral M1 phenotype compared to sphere CNPs.

Treatment with sphere CNPs, but not cube CNPs, significantly increases the recruitment of monocytes and dendritic cells to the airspace when compared to the absence of sphere CNPs treatment. Because dendritic cells are a primary antigen presenting cell, sphere CNPs elicit greater dendritic cell mediated T cell activation. The percentage of nanoparticle+ monocytes, dendritic cells, and neutrophils is significantly greater for sphere CNPs treatment versus cube CNPs treatment, suggesting that shape-specific CNPs are differentially taken up by immune cells. Treatment with either sphere or cube CNPs leads to the recruitment of important immune cell populations to the RSV-infected airways. Additionally, cube CNPs are taken up with greater frequency by alveolar macrophages relative to other inflammatory cells recruited to the airspace following RSV infection. Alternatively, sphere CNPs are taken up by alveolar macrophages, monocytes and neutrophils to a similar extent. This is a critical feature of cube CNPs that make it ideally suited for influencing macrophage activation whilst avoiding enhanced uptake and activation of inflammatory cells such as neutrophils, which are associated with RSV-mediated inflammation.

Alveolar macrophages are sentinel immune cells in the lung that are important for regulating immune responses. It has been found that CNPs effectively induce alveolar macrophage that presumably activate the adaptive immune response to help clear respiratory viruses and/or diseases, such as RSV disease, and/or other infections that cause pathologic immune responses. This is of critical importance, as infants have a prolonged course of RSV infection.

In certain embodiments, the shape-specific CNPs are included in a composition, such as a pharmaceutical composition. For example, in certain embodiments, the CNPs are combined with other drugs or vaccines as adjuvants to achieve better therapeutic outcomes in RSV disease or other infections with similar pathologies. In certain embodiments, the pharmaceutical composition includes a pharmaceutical carrier. Suitable pharmaceutical carriers include those known in the art, such as, but not limited to, a solvent (e.g. an alcohol), a polymer, a nanoparticle, a liposome, a lipoprotein, a gel, a sugar or sugars, protein or other matrix, or carriage device.

The invention provides methods of treating patients suffering from, or suspected of suffering from, respiratory viruses and/or diseases, such as RSV disease, and/or other infections that cause pathologic immune responses. The methods include in vitro and/or in vivo methods of treating the virus or disease, e.g., RSV disease. Administering shape-specific CNPs according to the invention includes any act that provides shape-specific CNPs to a patient in a way that the shape-specific CNPs function for their intended purpose. The shape-specific CNPs are administered in an amount or dosage that is sufficient to modulate macrophage phenotypes along the spectrum of M1 and M2 phenotypes to combat respiratory viruses and/or diseases, such as RSV disease, and/or other infections that cause pathologic immune responses, and/or an amount or dosage that is sufficient to modulate ROS and RNS, and immune cell activation induced by the respiratory virus or disease, e.g. RSV infection.

The invention includes methods of treating respiratory viruses and/or diseases, such as RSV disease, and/or other infections that cause pathologic immune responses, which include administering to a patient a pharmaceutical composition containing sphere CNPs, cube CNPs, or a combination, blend or mixture thereof. In alternate embodiments, the sphere CNPs and cube CNPs are administered at the same time or administered at different times. The pharmaceutical composition is administered using various conventional routes known in the art. Non-limiting examples of routes of administration include, but are not limited to, intravenous, injection, e.g., directly into the region or target area to be treated, oral, sublingual, buccal, transdermal, and nasal.

In certain embodiments, the pharmaceutical composition is a parenteral dosage form. In certain other embodiments, the pharmaceutical composition is an oral dosage form. In still other embodiments, the pharmaceutical composition comprises a tablet, capsule, dry powder, gel, film, suspension, solution or combination.

In certain embodiments, the total amount of the pharmaceutical composition is administered to the patient in a single dose, over a relatively short period of time, or using a treatment protocol in which multiple doses are administered over a more prolonged period of time. Concentration and quantity required in a treatment protocol depends on various factors including, but not limited to, age and general health of the patient, as well as the route of administration.

In other embodiments, the shape-specific CNPs, e.g., sphere CNPs and/or cube CNPs and/or rod CNPs, are administered to the patient using a single or multiple medical implant device(s) and/or a biodegradable and/or biocompatible matrix composition for the controlled release of the shape-specific CNPs in the body of a patient. In certain embodiments, the medical implant device(s) and/or matrix composition is positioned at or near a target location within the patient body, e.g., the site of the RSV disease, or other infection that causes a pathologic immune response.

In still other embodiments, the shape-specific CNPs, e.g., sphere CNPs and/or cube CNPs and/or rod CNPs, are administered to the patient by an ex-vivo application, such as, cell priming for cell therapy, to treat the RSV disease or other infections that cause pathologic immune responses.

### EXAMPLES

Experiments were conducted to assess the potential of cube and sphere-shaped CNPs to modulate ROS and RNS, and immune cell activation induced by RSV infection in vitro and in vivo. The in vitro results reveal a shape-dependent effect of CNPs in modulating ROS and RNS. Specifically, cube CNPs significantly reduced RSV-induced ROS levels without affecting RNS levels, while sphere CNPs increased RSV-induced RNS levels with minimal effect on ROS levels. Cube CNPs drove an MI phenotype in RSV-infected macrophages in vitro by increasing macrophage surface expression of CD80 and CD86 with a concomitant increase in TNFα and IL-12p70, while simultaneously decreasing M2 CD206 expression. Intranasal administration of sphere and cube-CNPs was well-tolerated with no observed toxicity in adult BALB/c mice. Notably, cube CNPs preferentially accumulated in murine alveolar macrophages and induced activation, avoiding enhanced uptake and activation of inflammatory cells such as neutrophils, which are associated with RSV-mediated inflammation. In conclusion, there is evidence that CNPs modulate macrophage polarization and innate cellular responses in a shape-dependent manner during RSV infection.

### INTRODUCTION

Antioxidant cerium oxide nanoparticles (CNPs) in various shapes were synthesized, and it was shown that the ROS-scavenging activity of CNPs mitigates oxidative stress-induced calcification in patient-derived primary valve interstitial cells. The results also showed that such ROS-scavenging activity of CNPs is dependent on their shape. Similar to CNPs, gold and other metal oxides such as iron oxide, titanium oxide can be synthesized into various shapes such as sphere, cube, and rod. Nanoparticle shape influences cellular uptake, as demonstrated in the art by the enhanced specificity of cancer cell lines for rod-shaped ligand-coated nanoparticles compared to their spherical counterpart. Similarly, in the art, iron oxide and gold nanoparticles have shown shape-specific differential effects on uptake, cytokine production, and inflammasome-activating capacity in macrophages in vitro and in mice. However, there is limited literature directly correlating the effect of nanoparticle shape alone on ROS-scavenging bioactivity or immune cell response. Given the importance of balanced levels of reactive species for mounting robust immune response to contain viral spread without eliciting tissue damage and immunopathology, the potential of shape-specific CNPs in modulating macrophage phenotype and innate cellular responses in RSV disease was evaluated. To assess shape-dependent redox and macrophage modulation activity of CNPs, sphere and cube-shaped nanocrystalline CNPs were synthesized to target resident lung immune cells. Here, the potential of cube and sphere-shaped CNPs to modulate immune response against RSV infection in vitro and resident lung immune cells in vivo was assessed.

### RESULTS AND DISCUSSION

### Shape-Specific Cerium Oxide Nanoparticle (CNPs) Exhibit Distinct Physicochemical Properties

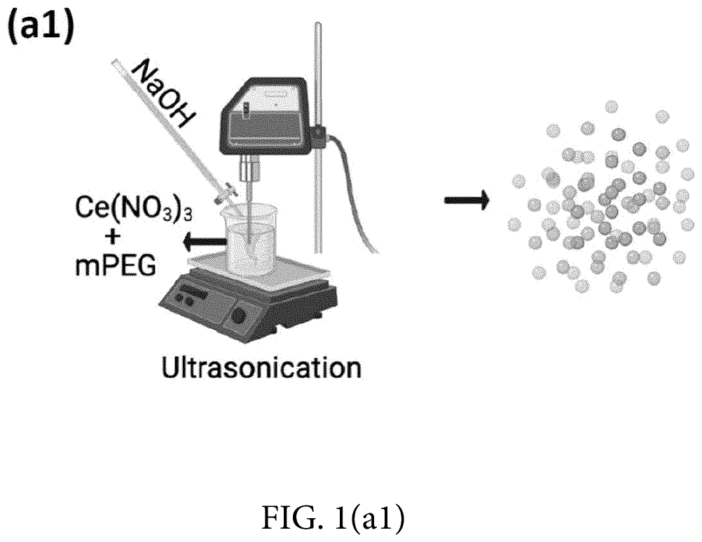

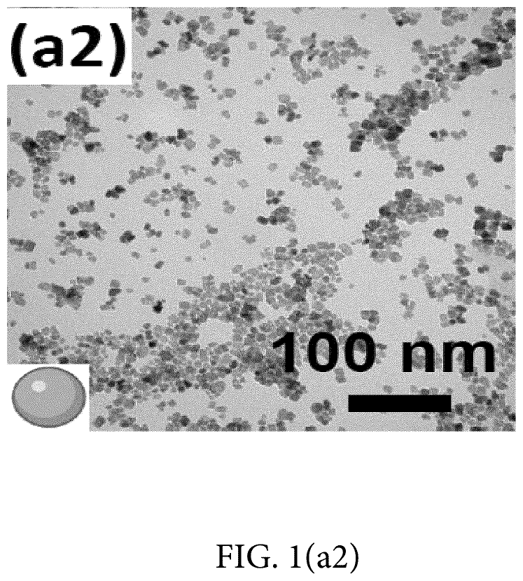

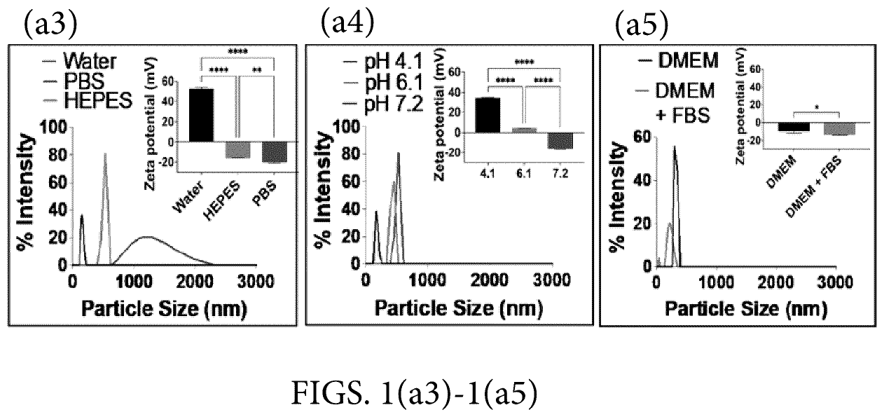

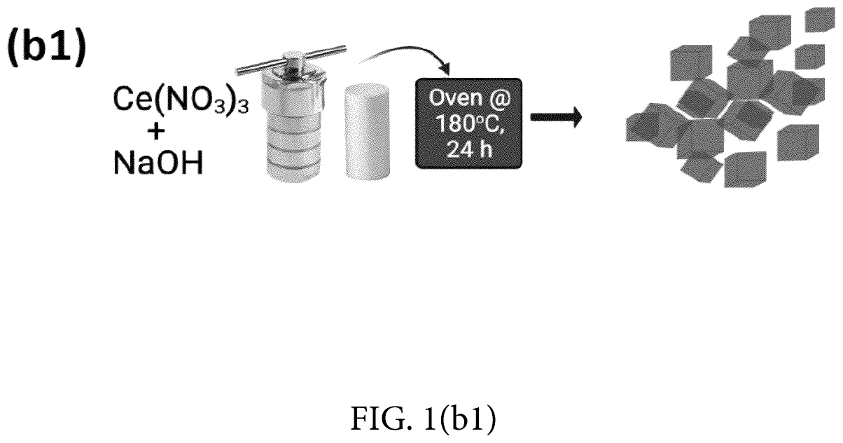

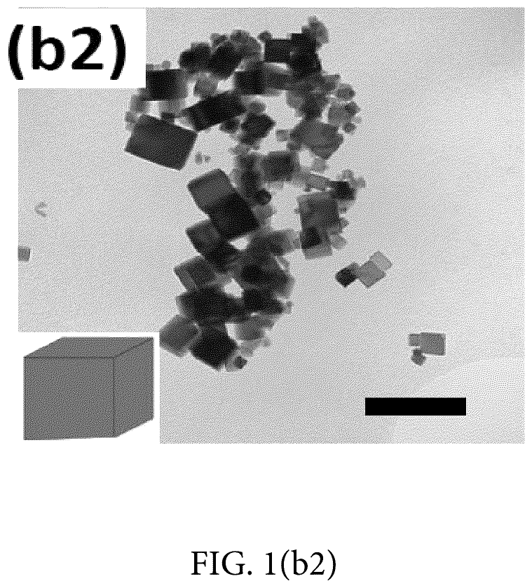

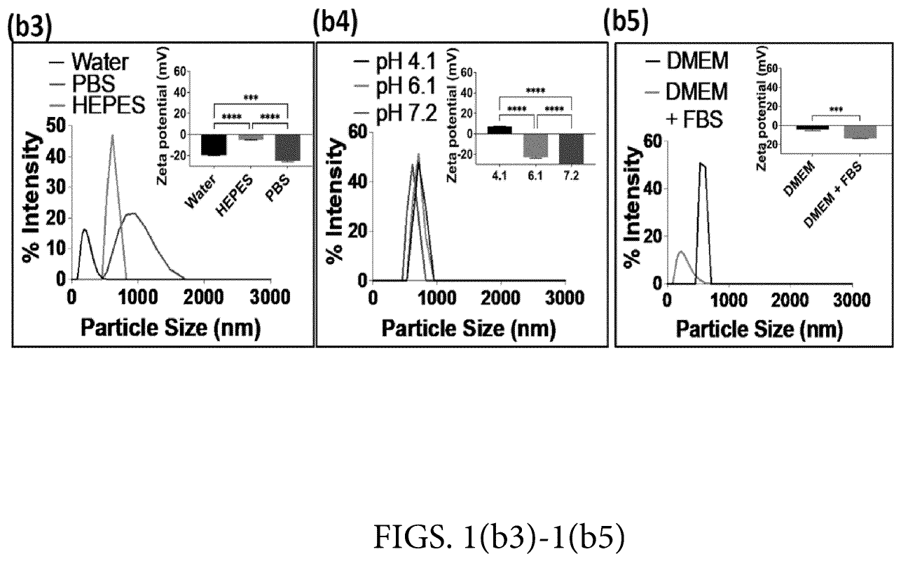

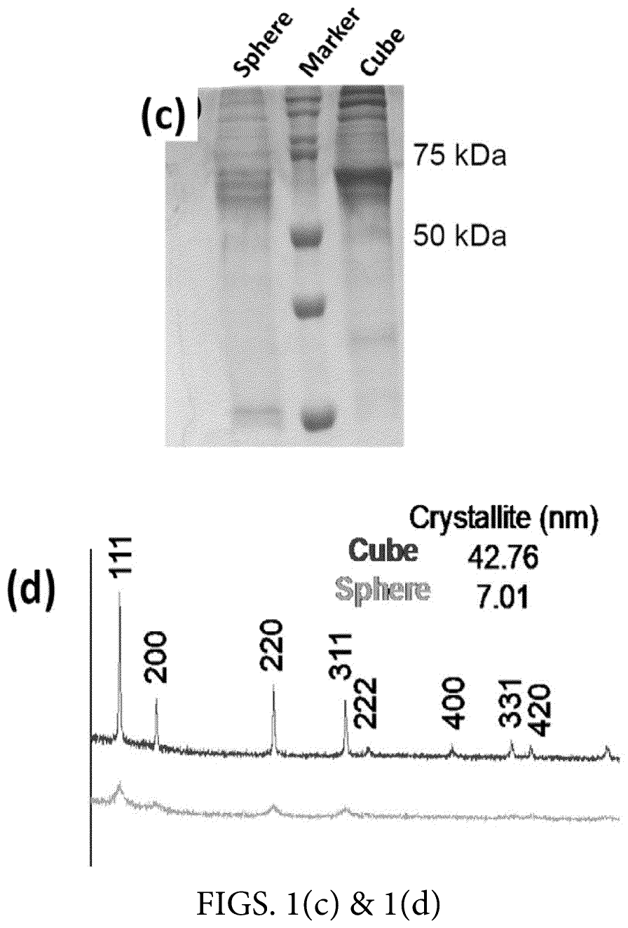

Differences in the shape, surface area, and dimensions of nanoparticles are known to affect various physicochemical properties such as catalytic performance of CNPs. To exploit shape-specific properties of CNPs, sphere and cube-shaped CNPs were synthesized by varying synthesis methodology for the reaction between cerium nitrate and sodium hydroxide. The sphere CNPs were synthesized by ultrasonication method (FIG. 1(a1)), whereby colloidal nanocrystals formed in the first step of the reaction adsorbed hydroxyl ions to form spherical nanocrystals. This was facilitated by weak surface adsorption of methoxy polyethylene glycol, (mPEG5000) added during the synthesis, which provided steric hindrance to prevent agglomerate formation. TEM micrographs confirmed the shape of sphere CNPs with an average diameter of 11.3 nm (FIG. 1(a2)). The cube CNPs were synthesized by hydrothermal method (FIG. 1(b1)) and their shape was confirmed by TEM (FIG. 1(b2)) with an average dimension of 19.3 nm. Formation of nanocube CNPs could be explained by higher temperatures achieved under high pressure for 24 h when the reaction was carried out in a hydrothermal reactor. This allowed oxidation of the intermediate nanocrystals such as nanorods and nanotubes, which further coarsened to reach stable crystal state forming cube morphology through Ostwald ripening.

To identify an optimum suspension medium for CNPs, the effect of 4-(2-hydroxyethyl)-1-piperazineethanesulfonic acid (HEPES) buffer (non-ionizable buffer system), phosphate buffered saline (PBS) (ionizable buffer system) and water on the hydrodynamic size and zeta potential of CNPs was assessed. Sphere CNPs showed average hydrodynamic size of 158±26.4 nm in water, 676.4±55.9 nm in HEPES, and 1227.6±77.9 nm in PBS, whereas cube CNPs showed an average hydrodynamic size of 208.8±3.62 nm, 561.16±72.8 nm, and 904.96±37.4 nm in water, HEPES, and PBS, respectively. Dynamic light scattering (DLS) measurements showed narrow size distribution for sphere (FIG. 1(a3)) and cube (FIG. 1(b3)) CNPs in HEPES buffer and water, while both CNPs showed a broad particle size distribution in PBS, suggesting an important role of ionic strength of the media in determining aggregation behavior of sphere and cube CNPs. Indeed, it has been shown that the long-range electrostatic forces and short-range hydration interactions play a critical role in the stability of CNPs in a suspension, where an increase in the ionic strength or pH resulted in reversible aggregation of CNPs. Both sphere and cube CNPs showed negative zeta potential in HEPES and PBS. On the other hand, sphere CNPs showed positive zeta potential and cube CNPs showed negative zeta potential in water (inset graphs, FIGS. 1(a3), 1(b3)). These shape-dependent differences in the surface charge in water could be attributed to the presence of PEG coating on the surface of sphere CNPs.

Since both sphere and cube CNPs showed less aggregation in HEPES buffer compared to PBS, the effect of pH (pH 4.1, 6.1 and 7.4) on CNP size and zeta potential using HEPES buffer was assessed. At pH 4.1, sphere CNPs showed high positive zeta potential and hydrodynamic size of 165.6±14.3 nm (FIG. 1(a4)). Since the isoelectric point of CNPs is about 6.5, sphere CNPs showed larger aggregates and zeta potential close to zero at pH 6.1, while their zeta potential turned negative at pH 7.2. The cube CNPs showed minimal effect of pH with consistently similar hydrodynamic size of 699.3±23.1 nm at all pH, while their zeta potential switched from 6.9±0.5 to −31.2±0.6 as pH increased from 4.1 to 7.2 (FIG. 1(b4)).

The extent of protein adsorption was determined by measuring size and zeta potential of CNPs in DMEM with and without (10% v/v) fetal bovine serum. Both, sphere (FIG. 1(a5)) and cube CNPs (FIG. 1(b5)) showed significant reduction in average particle size as well as zeta potential in the presence of serum. This could be attributed to stabilization of CNP suspension by adsorption of serum proteins, which would be beneficial under physiological conditions. Quantification of protein adsorption by SDS-PAGE revealed higher protein adsorption in cube CNPs compared to sphere CNPs (FIG. 1(c)). Reduced protein adsorption on sphere CNPs may be due to the presence of PEG coating on the surface of sphere CNPs and thus, steric hindrance for protein adsorption.

Catalytic power of CNPs is known to be shape-specific because of differences in highly reactive exposed crystal planes. It has been shown that crystalline cube CNPs contain more highly reactive exposed crystal planes compared to other shapes. Therefore, crystalline properties of CNPs was characterized using XRD. Sphere CNPs showed a moderately crystalline phase whereas cube CNPs showed a highly crystalline phase as evident from sharp peaks in XRD spectra (FIG. 1(d)). Crystallite calculation through the Scherrer equation showed four-fold higher crystallite phase for cube CNPs compared to sphere CNPs indicating larger size and more stable nanocrystal form for cube CNPs as compared to sphere CNPs.

### Shape-Specific CNPs are Cytocompatible and Readily taken up by J774A.1 macrophages In Vitro

To determine the potential cytotoxicity of sphere and cube CNPs, the viability of J774A. 1 macrophage cells exposed to increasing concentration of CNPs was assessed using an Alamar Blue assay. Metabolic activity of macrophages with CNP treatment was compared with macrophages without CNP treatment to calculate percentage viability. Both sphere and cube CNPs demonstrated greater than 70-80% viability for doses up to 300 μg/m (FIG. 2(a)). To investigate if CNPs were taken up by J774A.1 macrophages, cellular uptake of FITC-labeled CNPs was analyzed by confocal imaging, after 6 h of incubation, the sphere CNPs (FIGS. 2(b1)-2(b5)) and cube CNPs (FIGS. 2(cl)-2(c5)). Confocal image analysis further showed significantly higher mean fluorescence intensity (MFI) for cube CNPs compared to sphere CNPs confirming qualitative observations and indicating higher macrophage uptake of cube CNPs (FIG. 2(d)). Although two types of CNPs in this study were below 0.5 μm in size, cube-CNPs showed higher crystallinity and therefore, could exhibit greater catalytic activity and reactivity with macrophages relative to sphere CNPs.

CNP shape triggers differential ROS and RNS levels in RSV-infected macrophages

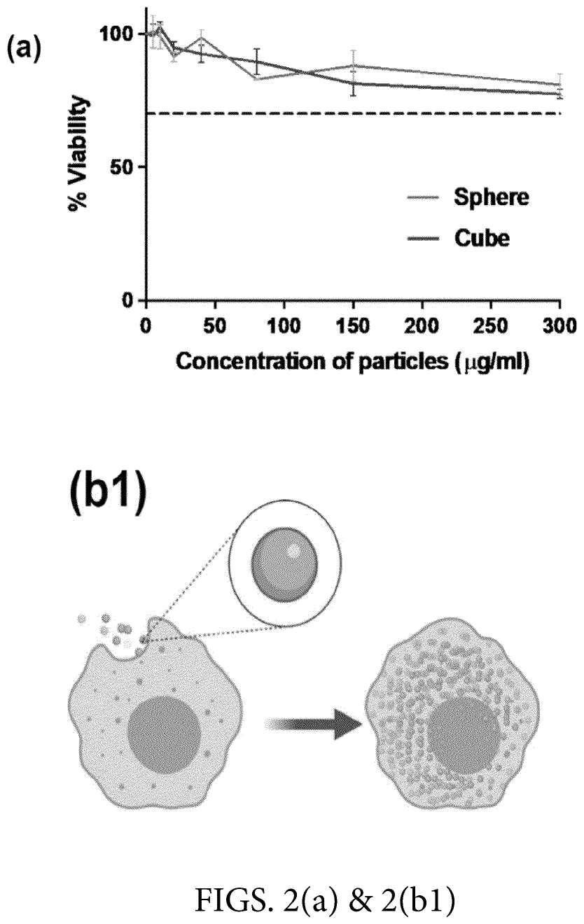

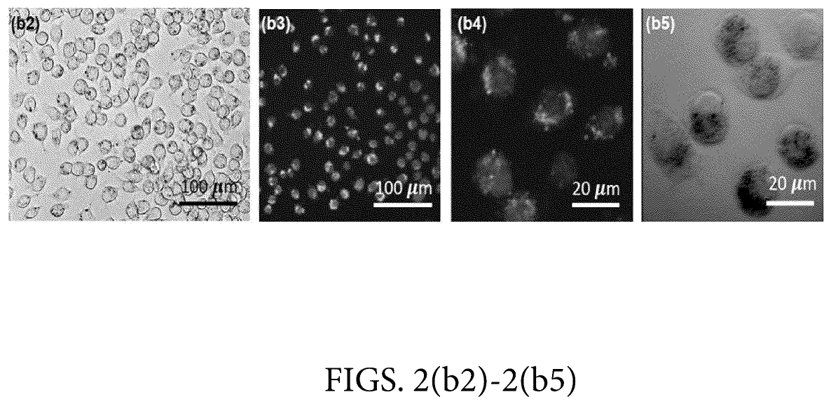

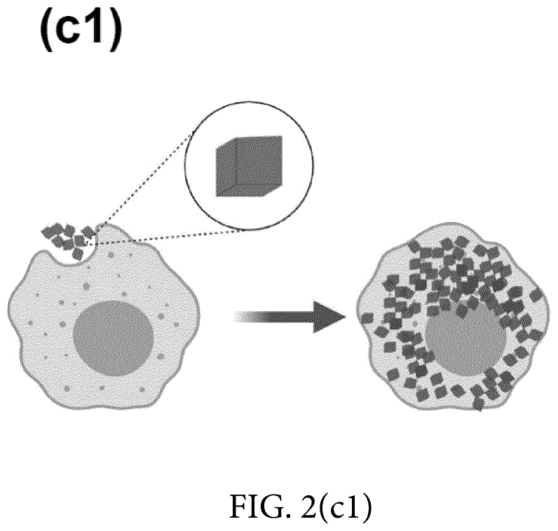

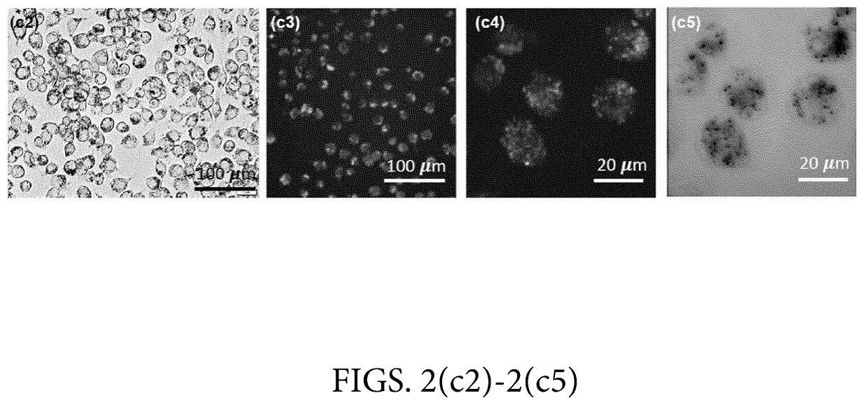

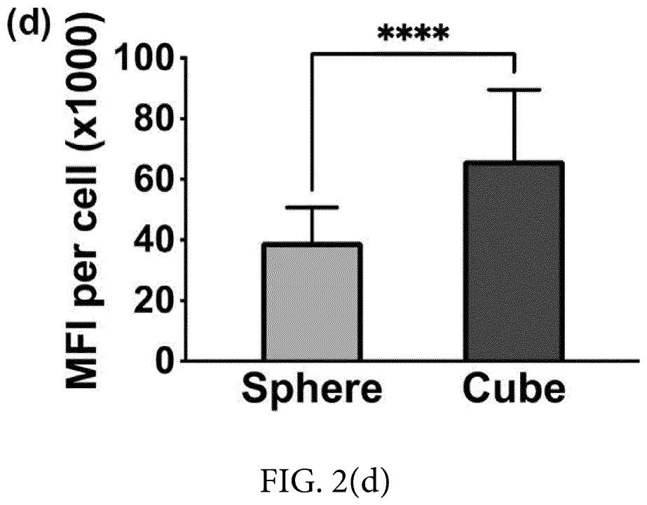

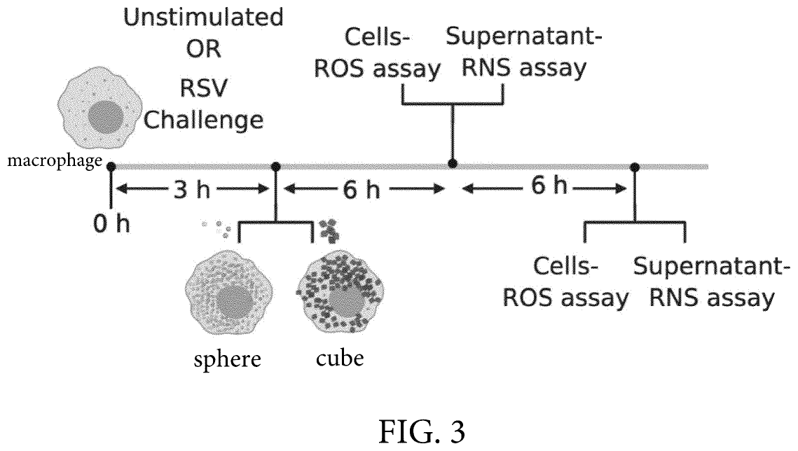

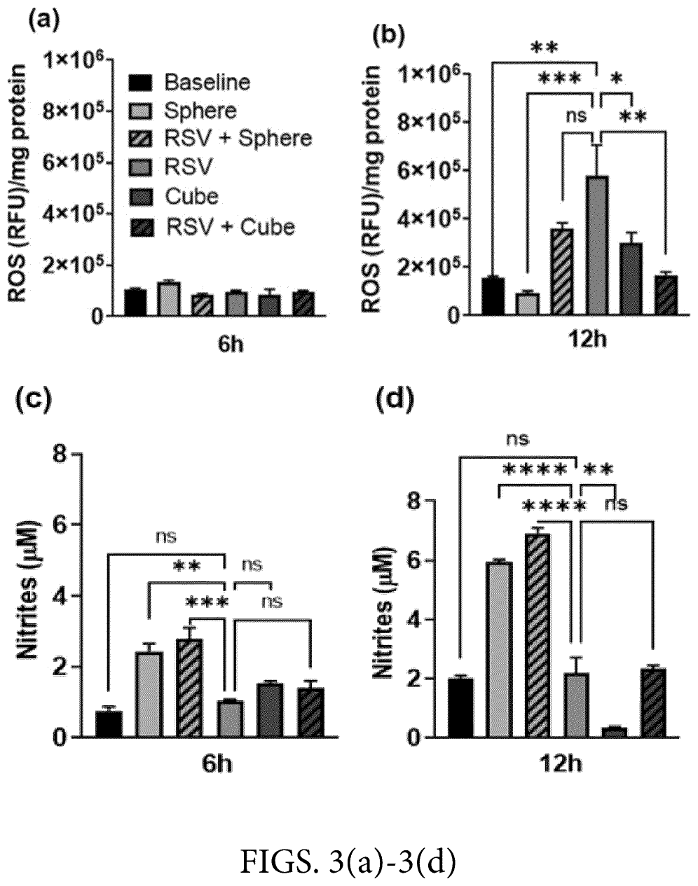

Increased ROS level is one of the hallmarks of RSV infection, which leads to tissue damage and airway inflammation. To evaluate the potential of CNPs to alter redox microenvironment, an in vitro assay was developed where virus-free J774A. 1 macrophages or macrophages infected with RSV L19 were subsequently treated with sphere or cube CNPs (FIG. 3). The ability of sphere and cube CNPs to modulate reactive oxygen and nitrogen species in J774A 1 macrophages in the absence or presence of RSV infection was measured. ROS levels were measured in live cells using CellROX® fluorogenic microplate assay. The CellROX® reagent is weakly fluorescent and changes to bright green color under oxidized state in the presence of ROS. Neither sphere nor cube CNPs significantly altered ROS levels in uninfected and RSV-infected macrophages after 6 h nanoparticle treatment (FIG. 3(a)). After 12 h, RSV infection induced significantly higher ROS production (˜6×105 RFU/mg protein, red bar) in infected macrophages compared to uninfected macrophages (black solid bar) (FIG. 3(b)). Interestingly, 12 h treatment of RSV-infected macrophages with cube CNPs (blue striped bar) significantly reduced RSV-induced increase in ROS levels. This may suggest the potential of cube CNPs to reduce tissue damage through reduction of ROS levels in RSV-infected macrophages. Although not statistically significant, a similar trend was observed with sphere CNP treatment in RSV-infected macrophages (green striped bar), highlighting the ability of both sphere and cube CNPs to decrease RSV-induced ROS production. In a previous study, sphere CNPs scavenged acute ROS generated by hydrogen peroxide treatment in valvular interstitial cells in a dose-dependent manner, whereas pretreatment of cube CNPs to hydrogen peroxide challenge prevented increases in ROS levels. Such free radical scavenging activity of CNPs is exerted through their superoxide dismutase-mimetic mechanism. The ratio of Ce3+ to Ce4+ on CNPs surface could be one of the differentiators between sphere and cube CNPs since a greater ratio indicates more oxygen vacancies and therefore, more acute ROS-scavenging activity.

Reactive nitrogen species (RNS) are known to be the fulcrum that shifts the macrophage polarity from pro-inflammatory (M1) to regenerative (M2) states. Optimal extracellular RNS level is important to enable cytotoxic activity of macrophages. Unlike ROS generated within the cytoplasm of the macrophages, RNS is generated extracellularly from rapid diffusion of nitric oxide (NO) out of cell membrane. Nitric oxide reacts with other oxygen species in the extracellular medium producing RNS, which are relatively more stable compared to short-lived nitric oxide. Such extracellular RNS, together with induced nitrous oxide synthase (iNOS) enzyme, provide protection and eradicate pathogens such as mycobacterium tuberculosis. RSV infection is known to alter homeostatic levels of NO and RNS primarily through iNOS induction. Therefore, we measured the extracellular RNS levels using Griess reagent in CNP-treated virus-free and RSV-infected macrophages (FIGS. 3(c)-3(d)). At 6 h, sphere CNP treatment of RSV-infected macrophages (green striped bar) significantly increased RSV-induced RNS levels (red solid bars) (FIG. 3(c)). These higher RNS levels were sustained 12 h post-RSV infection in sphere CNP-treated RSV-infected macrophages (FIG. 3(d)). Cube CNP treatment (blue striped bar), however, did not alter RSV-induced RNS levels, further highlighting shape-driven differential responses in modulating extracellular RNS levels in RSV-infected macrophages. Such shape-driven differential response in RNS modulation was also evident in virus-free macrophages treated with CNPs for 12 h. Specifically, sphere CNPs increased and cube CNPs reduced RNS levels significantly in virus-free macrophages compared to untreated RSV-infected macrophages at 12h (FIG. 3(d)).

Collectively, cube CNPs reduced RSV-induced ROS levels significantly without affecting RNS levels while sphere CNPs increased RSV-induced RNS levels with minimal effect on ROS levels.

### Shape of CNPs Alters Macrophage Phenotypes in Presence of RSV Infection In Vitro

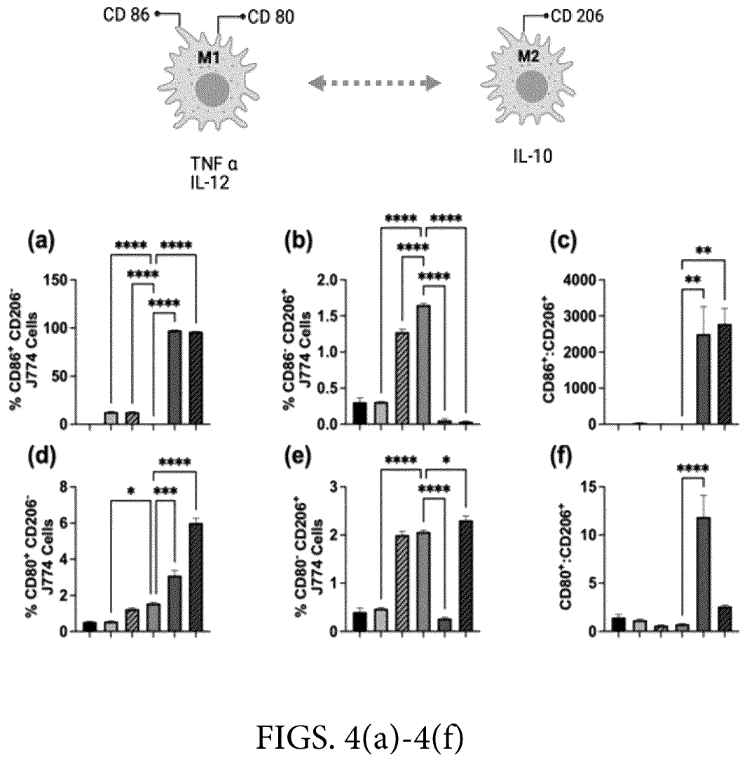

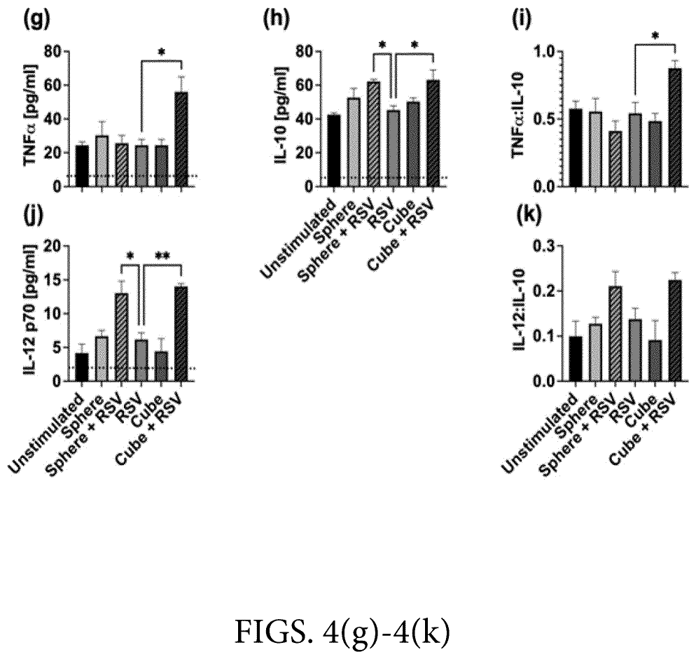

The reduction of ROS levels and optimal RNS levels are beneficial to prevent tissue damage in lungs. Therefore, to investigate the impact of differential reactive species generation by CNPs on RSV-infected macrophage phenotype, surface markers and cytokines secreted by virus-free and RSV-infected macrophages treated with sphere and cube CNPs were characterized. To evaluate the potential of CNPs to polarize macrophage phenotype, surface expression of M1 markers (CD86 and CD80) and the M2 marker (CD206) were measured 6 h post-CNP treatment via flow cytometry. RSV infection alone (red bars) failed to increase the frequency of M1 macrophages (FIGS. 4(a)-4(b)), while treatment of uninfected and RSV-infected macrophages with sphere and cube CNPs increased the frequency of M1 CD86+ CD206− J774A.1 macrophages. The increased MI macrophage phenotype (CD86+ CD206−) was predominantly driven by the CNPs irrespective of infection status as reflected by the ˜12% increase in sphere-treated macrophages and ˜96% increase in cube-treated macrophages (FIG. 4(a)). On the other hand, sphere and cube CNPs reduced the frequency of M2 CD86−CD206+ macrophages in both uninfected and RSV-infected macrophages relative to RSV infection alone (FIG. 4(b)). These results suggest that CNP shape influences RSV-associated macrophage activation with cube CNPs favoring a more anti-viral Ml phenotype compared to sphere CNPs at 6 h post-treatment (FIG. 4(c)). Consistent with FIG. 4(a), the use of CD80 as an alternative M1 marker showed that cube CNPs increased the frequency of M1 macrophages, but sphere CNPs did not (FIG. 4(d)). Similarly, only Cube+RSV increased M2 CD80− CD206+ macrophages (FIG. 4(c)). A comparison of the CD80+ M1 to M2 phenotype showed greater frequency of the anti-viral M1 versus M2 macrophage phenotype with cube CNPs (CD80+:CD206+ ratio) when compared to RSV alone (FIG. 4(f)).

Given the ability of CNPs to alter the surface expression of M1 and M2 macrophage markers, we next assessed macrophage function by measuring cytokine production following treatment with sphere or cube CNPs in uninfected and RSV-infected macrophages. Consistent with the dominant M1 phenotype shown in FIGS. 4 (a)-4(f), cube CNPs led to a significant increase in the production of TNFα, an M1-associated cytokine in RSV-infected macrophages (FIG. 4(g)). Interestingly, both sphere and cube CNPs induced a modest increase in the M2-associated cytokine IL-10 (FIG. 4(h)), which functions to prevent excess inflammation. A ratio of TNFα to IL-10 (FIG. 4(i)) indicates that cube, but not sphere CNPs favored the anti-viral TNFα response at the 24 h time point in RSV-infected macrophages as compared to untreated macrophages (red solid bars). Cube CNPs also induced a significant increase in the production of IL-12p70, an additional MI-associated cytokine in RSV-infected macrophages relative to untreated macrophages (FIG. 4(j)). Unlike TNFα, an increase in IL-12p70 was also noted with sphere CNPs in RSV-infected macrophages (FIG. 4j)). Although non-significant, cube CNPs induced an increase in ratio of IL-12p70 to IL-10 in RSV-infected macrophages (FIG. 4(k)). Taken together, cube CNPs drove an MI phenotype by increasing macrophage surface expression of CD80 and CD86 with a concomitant increase in TNFα and IL-12p70, while simultaneously decreasing M2 CD206 expression.

CD80 and CD86 provide critical co-stimulation signals for the full activation and functional responses of T cells. The higher expression of CD80 and CD86 in cube CNP-treated macrophages is indicative of a phenotype shift aligned with the ability to effectively stimulate anti-viral T cells, which are known to protect against RSV infection. These data confirm that RSV infection favors an M2 macrophage phenotype in the J774A.1 mouse macrophage cell line. It has been shown that the M2 phenotype was overcome with the addition of anti-viral IFNγ resulting in reduced viral titers and greater iNOS. Similarly, in vivo work confirmed that IFNγ treatment of RSV-infected BALB/c mice significantly induced alveolar macrophage activation and reduced viral titers. However, the use of inhaled IFNγ as a clinical therapeutic option is limited by the risk of inducing extensive inflammation in the infant airway. These data suggest that the use of cube CNPs offers a feasible alternative to activation of macrophages in a more controlled and balanced manner.

### Intranasal Administration of Shape-Specific CNPs is Safe

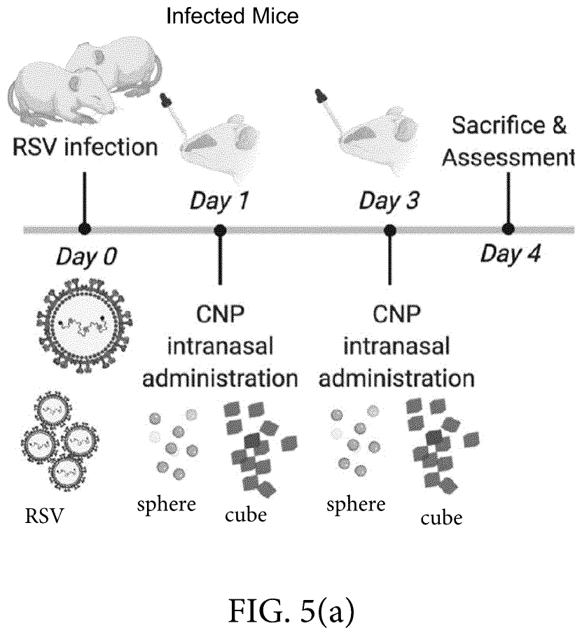

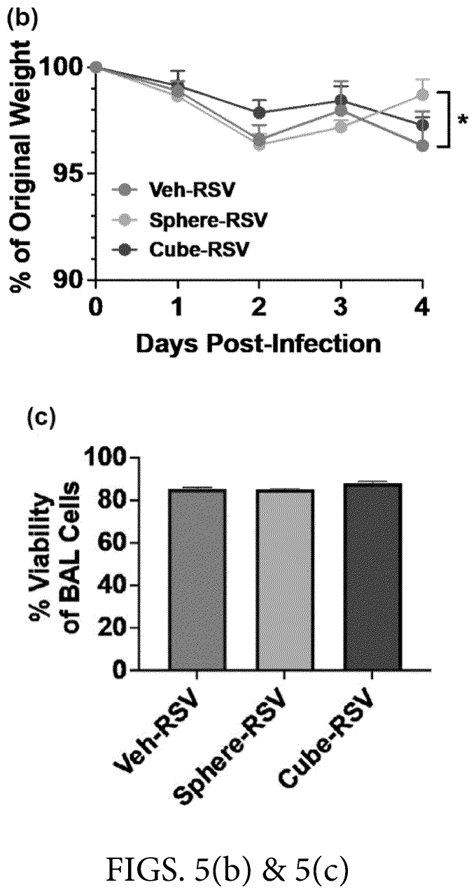

To translate the findings to an in vivo model and to assess CNP safety, adult mice were intranasally infected with RSV L19 and treated with two intranasal doses of sphere or cube CNPs at 1- and 3-days post-infection (FIG. 5(a)). As expected, mice in all 3 groups lost weight at 2 days post-infection, which was attributed to early MI cytokine release following RSV infection. By 4 days post-infection, mice that received sphere CNPs had lost significantly less weight than untreated mice and cube CNPs appeared to have less overall weight loss at each day through 4 days post-infection (FIG. 5(b)). Moreover, treatment with sphere or cube CNPs did not reduce the viability of cells retrieved from the bronchoalveolar lavage (BAL) fluid (FIG. 5(c)), suggesting that both sphere and cube CNPs are safe in mice in the doses given. Taken together, these results suggest that in vivo administration of both sphere and cube CNPs is safe at the doses used in these studies. It is important to note, however, that RSV-mediated disease pathology is primarily driven by the host immune response to RSV, rather than viral-mediated cellular destruction. Thus, CNPs provide a treatment option that is safe and effective to subvert excess inflammation.

### Shape of CNPs Leads to Differential Uptake by Immune Cells in Murine Lungs

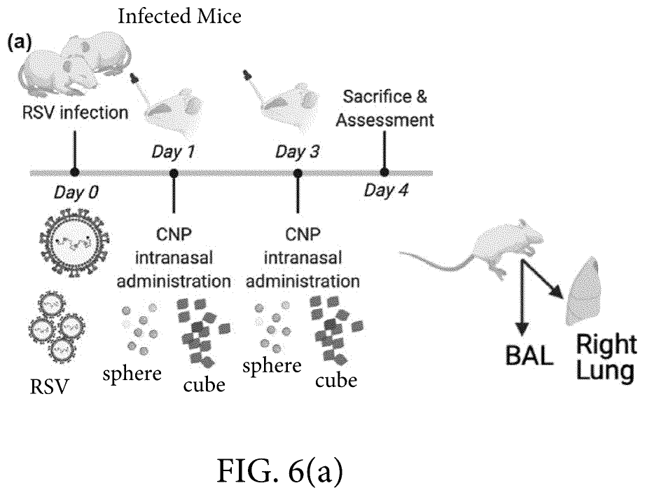

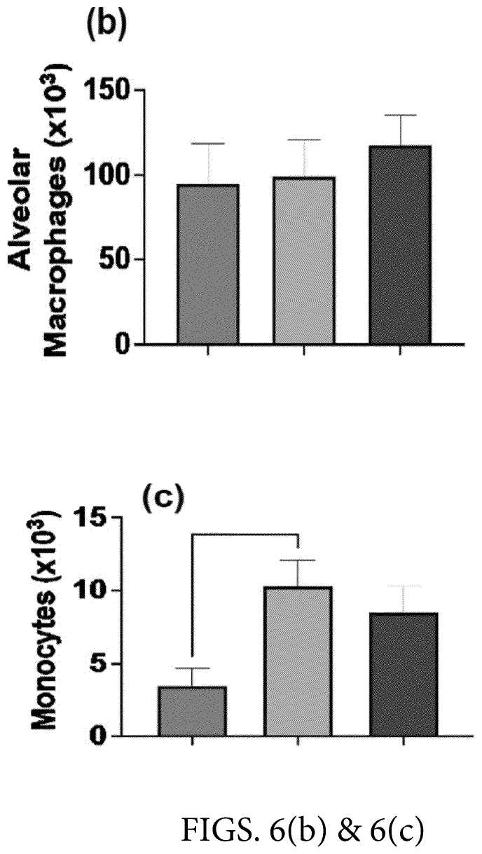

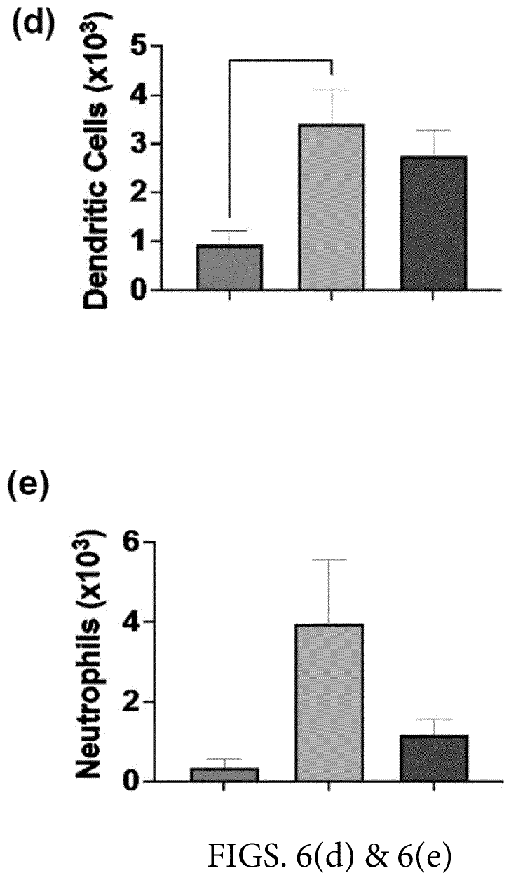

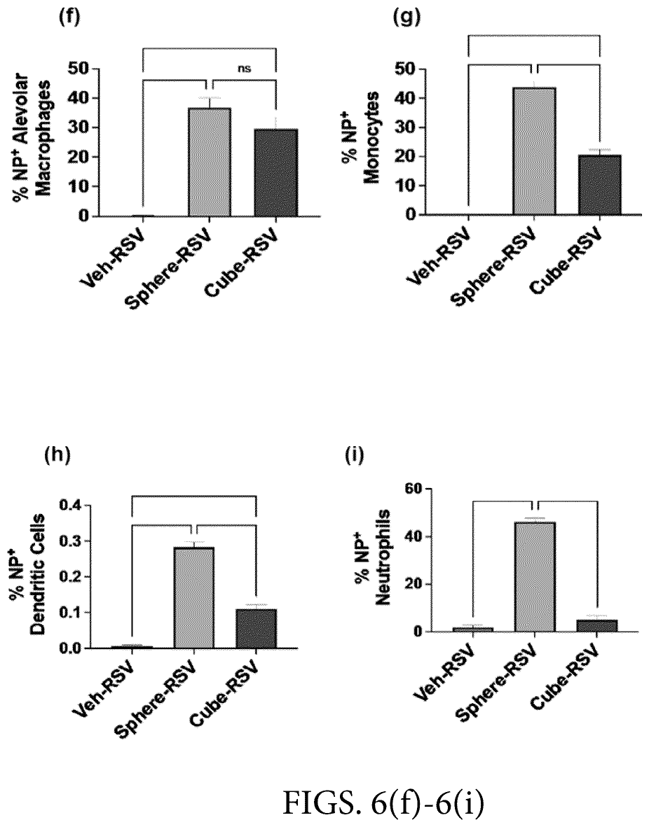

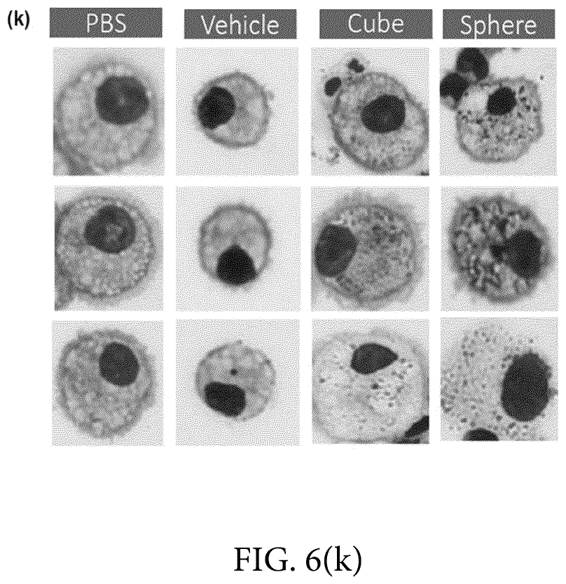

Innate immune cell infiltration into the BAL of RSV-infected animals following administration of either sphere or cube CNPs was assessed, as described in FIG. 6(a). Treatment with sphere or cube CNPs following intranasal RSV infection did not alter the number of alveolar macrophages in the airspace (FIG. 6(b)). However, treatment with sphere CNPs (greens solid bar), but not cube CNPs (blue solid bar), significantly increased the recruitment of monocytes (FIG. 6(c)) and dendritic cells (FIG. 6d)) to the airspace when compared to untreated animals. Because dendritic cells are a primary antigen presenting cell, these data suggest that sphere CNPs may elicit greater dendritic cell mediated T cell activation. Additionally, there was a non-significant trend toward increased neutrophil infiltration in sphere CNP-treated animals compared to untreated and cube CNP groups (FIG. 6(e)), suggesting a shape-dependent recruitment of inflammatory cells to the airway: lack of significance in this data is likely due to variability in the sphere CNP group. Using FITC-labeled CNPs, the uptake of nanoparticles by the various immune cell populations present in the BAL was assessed. The percentage of alveolar macrophages that phagocytosed nanoparticles was similar between sphere and cube CNP-treated animals (FIG. 6(f)), whereas the percentage of nanoparticle+monocytes (FIG. 6(g)), dendritic cells (FIG. 6(h)), and neutrophils (FIG. 6(i)) was significantly greater in mice that received sphere CNPs versus those that received cube CNPs, suggesting that shape-specific CNPs are differentially taken up by immune cells. Consistent with the flow cytometric analysis, images of CNP-treated macrophages indicated internalization of both sphere and cube CNPs (FIG. 6(k)). Taken together, these data indicate that treatment with either sphere or cube CNPs leads to the recruitment of important immune cell populations to the RSV-infected airways. Additionally, cube CNPs are taken up with greater frequency by alveolar macrophages relative to other inflammatory cells recruited to the airspace following RSV infection. Alternatively, sphere CNPs are taken up by alveolar macrophages, monocytes and neutrophils to a similar extent. This is a critical feature of cube CNPs that make it ideally suited for influencing macrophage activation whilst avoiding enhanced uptake and activation of inflammatory cells such as neutrophils, which are associated with RSV-mediated inflammation.

### CNP Treatment Activates Alveolar Macrophages

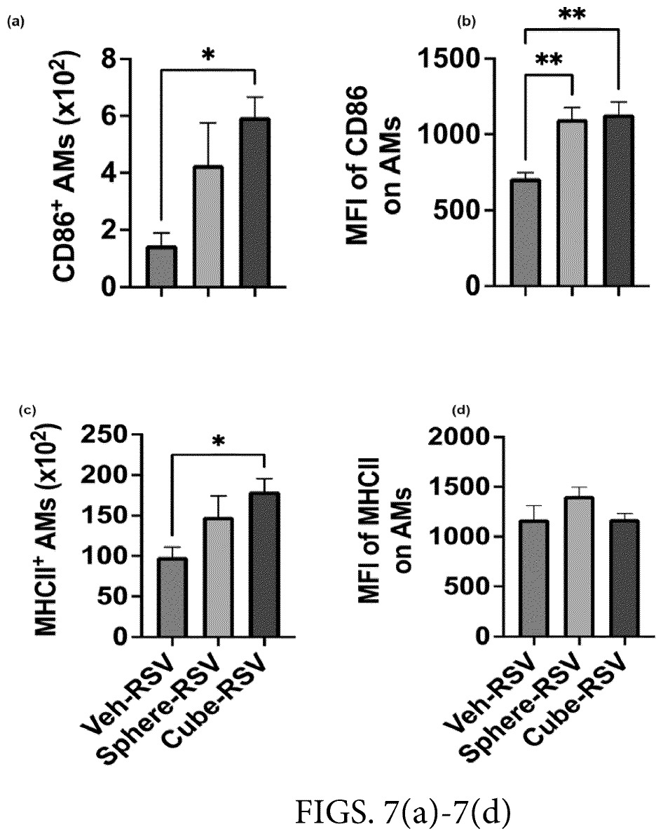

Alveolar macrophages are sentinel immune cells in the lung that are important for regulating immune responses. To assess if shape-dependent differential macrophage activation was associated with changes in cellular recruitment, the activation of alveolar macrophages in RSV-infected mice following sphere and cube CNP treatment was evaluated. Administration of cube CNPs, but not sphere CNPs, following RSV infection significantly increased the total number of CD86+ alveolar macrophages (FIG. 7(a)), while its MFI—a measure of CD86 expression on each cell—(FIG. 7(b)) was significantly increased following both sphere and cube CNP treatment when compared to untreated mice. Furthermore, treatment with cube CNPs, but not sphere CNPs, increased the total number of MHCII+ alveolar macrophages (FIG. 7(c)). Neither cube nor sphere CNP treatment significantly altered the MFI of MHCII surface expression (FIG. 7(d)) when compared to untreated RSV-infected mice. Taken together, these data suggest that cube, more than sphere CNPs alter the activation status of alveolar macrophages in vivo following RSV infection. MHC II is required for antigen presentation and CD86 is a critical co-stimulator molecule; both are required for the activation of RSV-specific B and T cells. Thus, these data suggest that CNPs effectively induce alveolar macrophage that will presumably activate the adaptive immune response to help clear RSV infection at later time points. This is of critical importance. as our previous studies have shown that infants. compared to adult mice. have a prolonged course of RSV infection.

We claim:

1. A pharmaceutical composition comprising:
shape-specific cerium oxide nanoparticles; and
a pharmaceutical carrier,
wherein the shape-specific cerium oxide nanoparticles are structured to switch between the Ce³⁺ and Ce³⁺ oxidation states to scavenge reactive oxygen species.

2. The pharmaceutical composition of claim 1, wherein the shape-specific cerium oxide nanoparticles have a shape selected from the group consisting of sphere, cube, rod, and combinations thereof.

3. The pharmaceutical composition of claim 1, wherein the pharmaceutical composition is in a dosage form selected from the group consisting of a parenteral dosage form, an oral dosage form, and combinations thereof.

4. The pharmaceutical composition of claim 3, wherein the dosage form is selected from the group consisting of a tablet, capsule, dry powder, gel, film, suspension, solution, and combinations thereof.

5. The pharmaceutical composition of claim 1, wherein the pharmaceutical carrier is selected from the group consisting of a solvent, a polymer, a nanoparticle, a liposome, a lipoprotein, a gel, one or more sugars, a protein, a carriage device, and combinations thereof.

6. The pharmaceutical composition of claim 1, wherein the shape-specific cerium oxide nanoparticles are structured to modulate macrophage phenotype and innate cellular responses in respiratory syncytial virus disease.

7. The pharmaceutical composition of claim 2, wherein the shape-specific cerium oxide nanoparticles in the shape of the sphere have an average diameter from about 8 to about 12 micrometer in dry form and 365 to about 557 micrometer in hydrodynamic form.

8. The pharmaceutical composition of claim 7, wherein the average diameter is about 11.3 nm.

9. The pharmaceutical composition of claim 2, wherein the shape-specific cerium oxide nanoparticles in the shape of the cube have an average dimension from about 24 to about 40 micrometer in dry form and 196 to 200 micrometer in hydrodynamic form.

10. The pharmaceutical composition of claim 9, wherein the average dimension is about 19.3 nm.

11. A method of treating respiratory syncytial virus disease or other infection that causes a pathologic immune response, comprising:
administering shape-specific cerium oxide nanoparticles to a patient, wherein the cerium oxide nanoparticles are structured to switch between the Ce³⁺ and Ce⁴⁺ oxidation states.

12. The method of claim 11, wherein the shape-specific cerium nanoparticles are selected from the group consisting of sphere cerium oxide nanoparticles, cube cerium nanoparticles, and combinations thereof.

13. The method of claim 11, wherein the administering step comprises administering a pharmaceutical composition comprising the shape-specific cerium oxide nanoparticles, and a pharmaceutical carrier.

14. The method of claim 11, wherein the administering step comprises applying ex-vivo the shape-specific cerium oxide nanoparticles to a patient.

15. The method of claim 13, wherein route of delivery of the pharmaceutical composition is by injection, oral, sublingual, buccal, transdermal, or nasal.

16. The method of claim 14, wherein the step of applying ex-vivo comprises cell priming for cell therapy.

17. A method of preparing shape-specific cerium oxide nanoparticles, comprising:
(i) preparing sphere-shaped cerium oxide nanoparticles, comprising:
synthesizing cerium oxide by an ultrasonication reaction between cerium nitrate and sodium hydroxide, comprising:
forming colloidal nanocrystals in a first step of the reaction, and
adsorbing hydroxyl ions to form spherical nanocrystals, or
(ii) preparing cube-shaped cerium oxide nanoparticles, comprising:
synthesizing cerium oxide by a hydrothermal reaction between cerium nitrate and sodium hydroxide, in a hydrothermal reactor, comprising:
oxidizing intermediate nanocrystals selected from the group consisting of nanorods and nanotubes; and
coarsening the nanocrystals to reach a stable crystal state forming cube morphology.

18. The method of claim 17, further comprising adding and surface adsorbing methoxy polyethylene glycol during the synthesizing step in (i) or (ii) to provide steric hindrance to prevent agglomerate formation.
